(19) United States
(12) Patent Application Publication
(10) Pub. No.: US 2006/0257946 A1
(43) Pub. Date: Nov. 16, 2006

(54) Serum biomarkers in ischaemic heart disease

(75) Inventors: Jian Ding, Dublin, CA (US); Lee Lomas, Foster City, CA (US)

Correspondence Address: CIPHERGEN c/o FOLEY & LARDNER LLP, 3000 K STREET NW, SUITE 500, WASHINGTON, DC, 20007, US

(73) Assignee: CIPHERGEN BIOSYSTEMS, INC

(21) Appl. No.: 10/559,687

(22) PCT Filed: Jun. 4, 2004
(86) PCT No.: PCT/US04/17624

§ 371 Date: May 11, 2006

Related U.S. Application Data
(60) Provisional application No. 60/476,176, filed on Jun. 6, 2003.

(51) Int. Cl. G01N 33/542; G01N 33/53

(52) U.S. Cl. 435/7.9

(57) ABSTRACT

Certain biomarkers and biomarker combinations are useful in a qualifying ischaemic heart disease status in a patient. A diagnostic methodology employing these biomarkers and combinations can distinguish between ischaemic heart disease and normal, as well as between cases of severe myocardial infarction versus mild myocardial infarction.

### BACKGROUND OF THE INVENTION

The present invention relates generally to the field of serum biomarkers in ischaemic heart disease (IHD). More particularly, the invention relates to serum biomarkers that can be used to classify myocardial infarct (MI) patients with enhanced specificity and sensitivity. The present invention also identifies biomarkers that are known proteins or fragments thereof.

Millions of patients are admitted to hospital annually with chest pain, including 10% to 15% that have suffered a myocardial infarction (MI). The current clinic method for assessing MI risk involves the detection of creatine kinase MB (CKMB) and myofilament proteins, such as cardiac troponins. Multiple markers are greatly sought after by clinicians to improve specificity and sensitivity in prognosis, diagnosis, staging of ischaemic heart diseases.

Elevated plasma fibrinogen is associated with increased risk for acute myocardial infarction, and stroke. Kannel et al., “Fibrinogen and risk of cardiovascular disease. The Framingham Study,” JAMA, 1183-6 (1987); Elwood et al., “Exercise, fibrinogen, and other risk factors for ischaemic heart disease. Caerphilly Prospective Heart Disease Study,” Br. Heart J. 69(2): 183-87 (1993). However, fibrinogen does not stand alone as a marker for cardiovascular event. For example, see Veres et al., “Relationship of anti-60 kDa heat shock protein and anti-cholesterol antibodies to cardiovascular events,” Circulation 106(22): 2775-80 (2002); Rallidis et al., “Prognostic value of C-reactive protein, fibrinogen, interleukin-6, and macrophage colony stimulating factor in severe unstable angina,” Clin. Cardiol. November; 25(11): 505-10 (2002).

Other diagnostic methods for IHD include catheterization coupled with fluoroscopy. This is an invasive procedure, involving introduction of a dye to which the patient may be sensitive.

It would be highly desirable, therefore, to have a biomarker or combination of biomarkers capable of non-invasively identifying IHD with sensitivity and selectivity. The literature on IHD diagnosis has not disclosed such a biomarker or biomarker combination, however.

### SUMMARY OF THE INVENTION

In accordance with the present invention, biomarkers and combinations of biomarkers are used to identify IHD. In one embodiment, a method for qualifying ischaemic heart disease status in a subject comprises measuring at least one biomarker in a sample from the subject, wherein the biomarker is galectin-3 or a galectin-3 fragment, and correlating the measurement with ischaemic heart disease status. Ischaemic heart disease status includes the presence or absence of disease, the degree of disease and the effectiveness of treatment of disease.

The inventive methodology additionally may comprise the managing of subject treatment based on the status. Such management may include ordering more tests, performing surgery, prescribing medication, and taking no further action. The methodology also may include measuring the at least one biomarker again, after subject management. The additional biomarker can be fibrinogen or a fibrinogen fragment, for example, and the measurement can be correlated with ischaemic heart disease status.

Pursuant to the invention, a subject sample of blood or a blood derivative, such as serum or plasma, is provided, proteins in the sample are fractionated, and fractions that contain galectin-3 or a galectin-3 fragment biomarker are collected and then captured on a surface of a substrate comprised of capture reagents that bind the biomarkers. Measuring of biomarkers includes detecting the presence or absence of the biomarker, quantifying the amount of marker(s), and qualifying the type of biomarker.

The substrate may be a SELDI probe that carries an adsorbent that captures the biomarkers. In one embodiment, the SELDI probe comprises a biospecific affinity reagent that binds the biomarkers, such as an antibody. Alternatively, the substrate is a microtiter plate comprising biospecific affinity reagents that bind the biomarker(s) and the biomarker(s) are detected by immunoassay. The biomarker(s) may be measured using a biochip array, particularly a protein chip array. The biomarker(s) are immobilized on the biochip or protein chip-array and may be measured by SELDI or an immunoassay.

The invention also contemplates a kit for detecting and diagnosing IHD. Kits within the invention comprise, for example, (i) an adsorbent attached to a substrate that retains one or more of the biomarkers according to the invention, and (ii) instructions to detect the biomarker(s) by contacting a sample with the adsorbent and detecting the biomarker(s) retained by the adsorbent. An inventive kit may further comprise a washing solution and/or instructions for making a washing solution.

More particularly, a kit according to the invention includes a capture reagent that binds a biomarker selected from galectin-3 and galectin-3 fragments, and instructions for using the capture reagent to detect the biomarker. The kit additionally may include a container comprising at least one of the biomarkers. In one embodiment, the capture reagent binds a plurality of the biomarkers. The capture reagent may be a SELDI probe. The kit may include an additional capture reagent that binds an additional biomarker selected from the group consisting of fibrinogen and fibrinogen, fragments, and this additional capture reagent may bind a plurality of the additional biomarkers. The additional capture reagent also may be a SELDI probe.

In a preferred embodiment, the capture reagent is a biospecific adsorbent, more particularly, an antibody. The capture reagent and the additional capture reagent may be an anti-galectin-3 antibody or an antitroponin I antibody. The additional capture reagent also may be an antifibrinogen antibody. The kit may further include a wash solution that selectively allows retention of bound biomarker to the capture reagent as compared with other proteins after washing.

In one embodiment, the kit contains a single capture reagent that binds both a biomarker selected from the group consisting of galectin-3 and galectin-3 fragments and an additional biomarker selected from the group consisting of fibrinogen and fibrinogen fragments, and instructions for using the capture reagent to detect both of the biomarkers. This kit additionally may include one or more containers that contain at least one of the biomarkers. In one embodiment, the kit includes at least one container comprising at least on of the biomarkers and at least one container comprising at least one of the additional biomarkers.

The kit may additionally contain written instructions for use of the kit in the diagnosis of ischaemic heart disease. These instructions may provide for contacting the biomarker(s) provided with the kit with the capture reagent and may describe the result of the contacting.

### BRIEF DESCRIPTION OF THE DRAWINGS

FIGS. 1A and 1B show biomarker patterns obtained for serum samples from severe and mild myocardial infarction, respectively.

FIG. 2 is a peptide fingerprint for the 7.8 kDa peak of FIG. 1A.

FIG. 3 shows the mass spectrum for fragmented ions from the 1985 peptide of FIG. 2.

FIG. 4 is a peptide fingerprint for the 6.7 and 8.9 kDa peaks of FIG. 1B.

FIG. 5 shows the mass spectrum for fragmented ions from the 1887 peptide of FIG. 4.

### DETAILED DESCRIPTION OF THE PREFERRED EMBODIMENTS

A key aspect of the present invention is the discovery of a series of biomarkers associated with IHD. In this context, a “biomarker” is an organic biomolecule, particularly a polypeptide or protein, which is differentially present in a sample taken from a subject having IHD as compared to a comparable sample taken from a normal subject that does not have IHD.

A biomarker is present differentially in samples taken from IHD and normal patients if it is present at an elevated level or a decreased level in samples of IHD patients as compared to samples of normal patients that do not have IHD. More particularly, a biomarker is a polypeptide that is characterized by an apparent molecular weight, as determined by gas phase ion spectrometry, and that is present in samples from IHD subjects in an elevated or decreased level, as compared to normal subjects. A biomarker is differentially present between two samples if the amount of the biomarker in one sample differs in a statistically significant way from the amount of biomarker in the other sample.

The biomarkers of the invention can be used to assess ischaemic heart disease status in a subject. The phrase “ischaemic heart disease status” subsumes, inter alia, the presence or absence of disease, the risk of developing disease, the stage of the disease, and the effectiveness of treatment of disease. Based on this status, further procedures may be indicated, including additional diagnostic tests or therapeutic procedures or regimens, such as balloon angioplasty, bypass surgery and/or treatment with one or more heart medications.

In some instances, a single biomarker is capable of identifying ischaemic heart disease with a sensitivity or specificity of at least 85%, whereas, in other instances, a combination or plurality of biomarkets is used to obtain a sensitivity or specificity of at least 85%. Thus, the biomarkers and combinations of biomarkers can be used to qualify ischaemic heart disease status in a subject or patient.

The biomarkers according to the invention are present in serum. The biological sample used according to the present invention, however, need not be a serum sample. Thus, a biological sample for qualifying ischaemic heart disease status may be a serum, plasma or blood sample, although serum samples are preferred.

Biomarkers are characterized by molecular weight. Moreover, certain biomarkers have been further characterized, and have been identified as galectin-3 or a fragment thereof. A fragment of galectin-3 includes a sequence of amino acids that is recognized by an epitope of an anti-troponin or anti-galectin-3 antibody. Fragments preferably have a molecular mass of at least about 200 Daltons, more preferably at least about 500 Daltons. In even more preferred embodiments, fragments have a molecular mass of at least about 800 Daltons, and most preferably at least about 1 Kilodalton. Suitable fragments for use in the present invention can be identified by enzymatically digesting galectin-3 and testing the resulting fragments for the ability to bind to an anti-galectin-3 antibody and/or an anti-troponin antibody. Fragments that bind either of these antibodies can be sequenced using techniques well-known in the art, although the sequence of the fragment is not needed to practice the invention. In order to practice the invention with a fragment from the enzymatic digest that is identified as binding antibody, all that is required is to subject to the fragment to mass spectrometry to determine its mass spectrograph.

Galectin-3 belongs to the galectin family, a highly conserved family of beta-galactoside binding mammalian lectins. These lectins share homologous carbohydrate recognition domains. Presently, more than 14 members have been identified and additional homologues are likely to be discovered. Rabinovich et al., “Role of galectins in inflammatory and immunomodulatory processes,” Biochim. Biophys. Acta. 1572(2-3): 274-84 (2002). Galectins have been implicated in different immunological processes, such as lymphocyte adhesion, cytokine production, cell growth regulation, apoptosis and central and peripheral immune tolerance. They play a role as putative modulators of immune surveillance, apoptosis, cell adhesion and chemotaxis. Research has shown that galectin-3 participates in development, immune response in oncogenesis, cell-to cell adhesion, and acute and chronic inflammation.

Galectin-3 has been associated with cell injury and regeneration in two types of acute real failure (ARF), ischaemic and toxic. Nishiyama et al., “Up-regulation of galectin-3 in acute renal failure of the rat,” Am. J. Pathol. 157(3): 815-23 (2000). Galectin-3 expression was markedly up regulated in both types of ARF, indicating that galectin-3 may play an important role in acute tubular injury and the following regeneration stage. To date, however, galectins have not been associated with ischaemic heart diseases, including MI, and interaction between galectins and CKMB and/or troponins has not been reported. Thus, galectin-3 and fragments thereof are unique markers for MI.

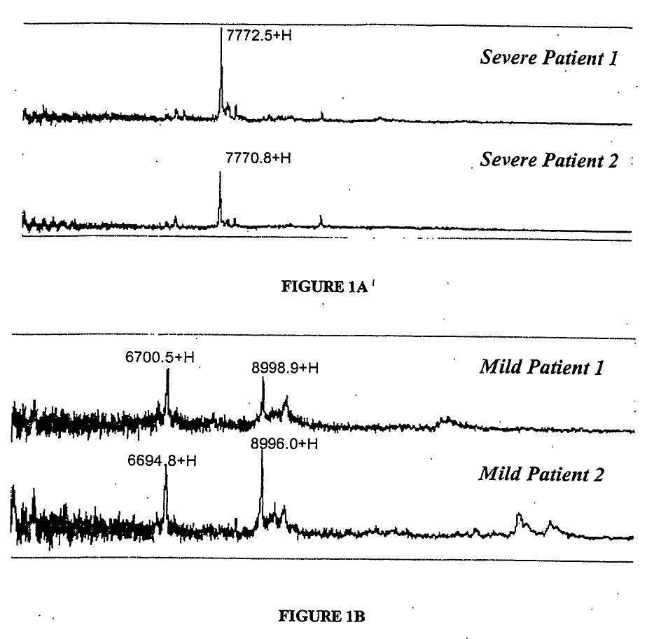

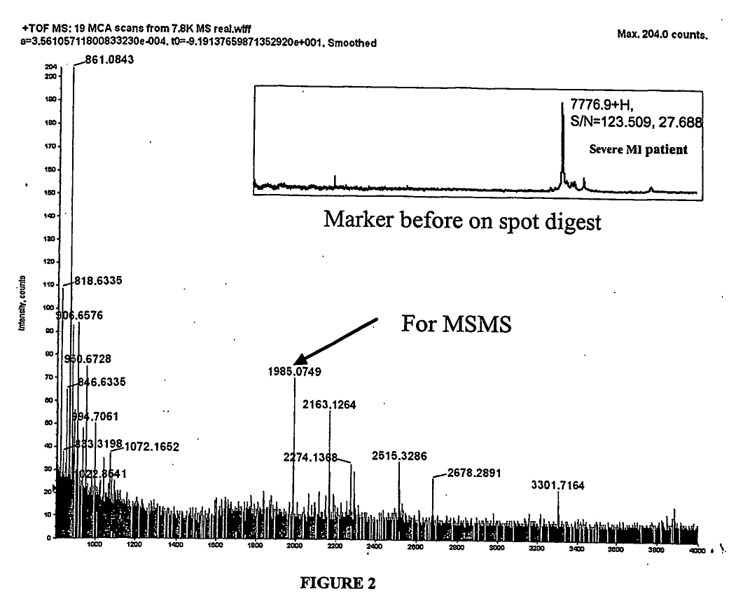

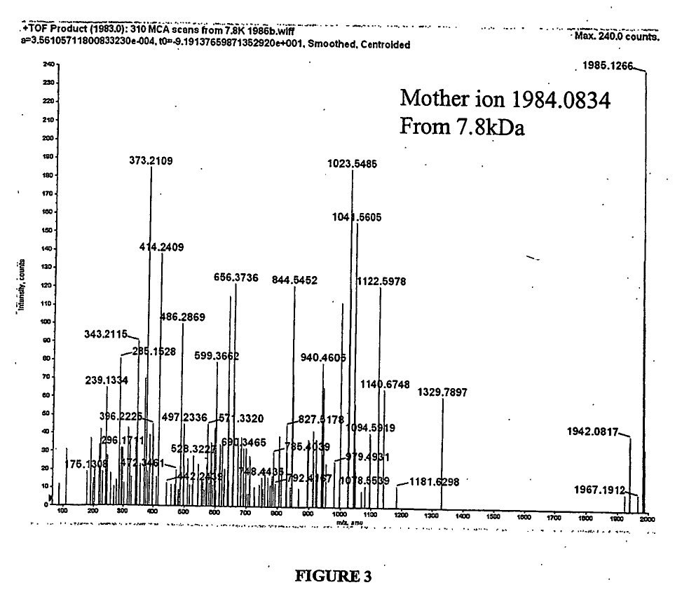

More particularly, a biomarker pattern for severe myocardial infarction has an intense protein peak at about 7.8 kDa, as shown in FIG. 1A. This peak was identified as having a sequence homologous to galectin-3. This identification was performed by enzymatically digesting the protein peak, mass sequencing the peptides obtained, and doing a database search to match the peptide fingerprints. The peptide fingerprint for the 7.8 kDa peak is shown in FIG. 2. Fragmented ions from the 1985 Da peptide are shown in FIG. 3, and were subjected to a Mascot database search, which confirmed that the 1985 Da peptide is galectin-3. Searches in other databases also identified the 7.8 kDa peak and the 1985 Da peptide fingerprint as being galectin-3.

Galectin-3 may be used alone to determine whether a patient has IHD, or it may be used in combination with or more other biomarkers indicative of IHD as determined according to the invention, including fibrinogen and fragments thereof.

Fibrinogen is a protein that is synthesized by liver and is converted to fibrin in the presence of the enzyme thrombin to form blood clots. As a global marker of inflammation, fibrinogen has been well studied as potential cardiovascular risk factors. Elevated plasma fibrinogen is associated with increased risk for acute myocardial infarction, and stroke, but is not a unique biomarker for IHD. It is useful in combination with galectin-3 and/or with other biomarkers according to the invention, in order to increase the sensitivity and/or selectivity of diagnosis and prognosis of IHD.

More particularly, a biomarker pattern for severe myocardial infarction has two intense protein peaks at about 6.7 and 8.9 kDa, respectively, as shown in FIG. 1B. These peaks were identified as having a sequence homologous to fibrinogen. This identification was performed by enzymatically digesting the protein peaks, mass sequencing the peptides obtained, and doing a database search to match the peptide fingerprints.

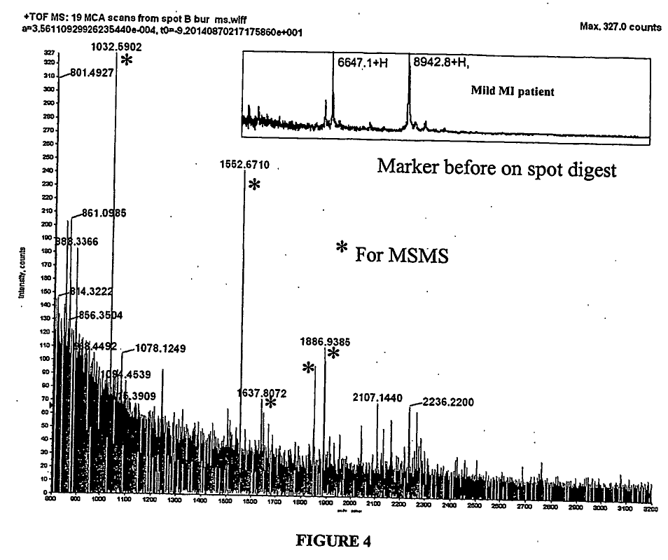

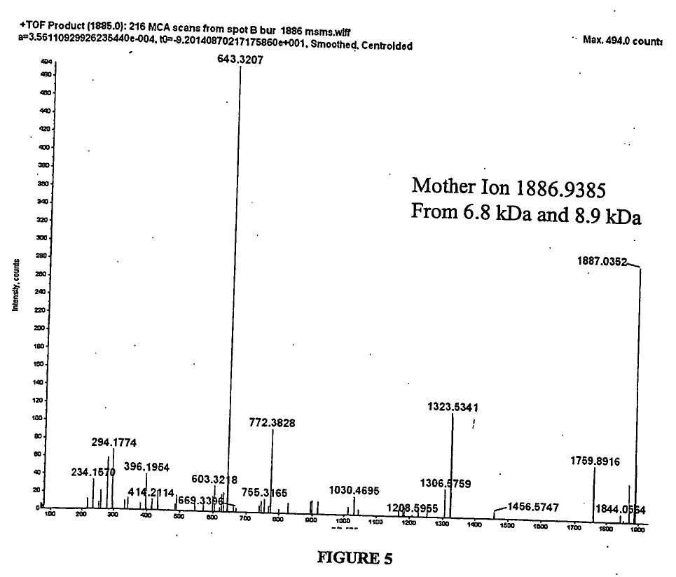

The peptide fingerprint for the 6.7 and 8.9 kDa peaks is shown in FIG. 4. All major peptides highlighted with * were analyzed. The peptides in the range of 2100 Da to 2300 Da are largely from trypsin and were excluded from mass sequencing. Fragmented ions from the 1887 Da peptide are shown in FIG. 5, and were subjected to a Mascot database search. A score greater than 16 for this Mascot search was highly significant.

The 1887 Da peptide had a score of 26 and good separation from other matches, thus confirming that the 1887 Da peptide is fibrinogen. Searches in other databases also identified the 1887 Da peptide fingerprint as being fibrinogen. Besides the 1887 Da peptide, all other highlighted peptides, including the 1032, 1552, 1637 and 1846 Da peptides were mass sequenced and shown to be homologous to fibrinogen. 10034] If desired, any of the galectin-3 and fibrinogen biomarkers can be sequenced, in order to obtain an amino acid sequence, but this is not required to practice the present invention. The biomarkers thus are characterized by molecular weight and/or by their known protein identities.

It has been found that proteins frequently exist in a sample in a plurality of different forms characterized by detectably different masses. These forms can result from pre-translational modifications, post-translational modifications or both. Pre-translational modified forms include allelic variants, splice variants, and RNA-editing forms. Post-translationally modified forms include forms resulting from, among other things proteolytic cleavage (e.g., fragments of a parent protein), glycosylation, phosphorylation, lipidation, oxidation, methylation, cystinylation, sulphonation and acetylation. The collection of proteins including a specific protein and all modified forms of it is referred to herein as a “protein cluster.” The collection of all modified forms of a specific protein, excluding the specific protein, itself, is referred to herein as a “modified protein cluster.” Also, modified forms of any biomarker of this invention themselves may be used as biomarkers. In certain cases the modified forms may exhibit better discriminatory power in diagnosis than the specific forms set forth here.

Modified forms of a biomarker can be initially detected by any methodology that can detect and distinguish the modified from the biomarker. A preferred method for initial detection involves first capturing the biomarker and modified forms of it, e.g., with biospecific capture reagents, and then detecting the captured proteins by mass spectrometry. More specifically, the proteins are captured using biospecific capture reagents, such as antibodies, interacting fusion proteins, aptamers, or Affibodies that recognize the biomarker and modified forms of it. This method may also result in the capture of protein interactors that are bound to the proteins or that are otherwise recognized by antibodies and that, themselves, can be biomarkers. Preferably, the biospecific capture reagents are bound to a solid phase. Then, the captured proteins can be detected by SELDI mass spectrometry or by eluting the proteins from the capture reagent and detecting the eluted proteins by traditional MALDI or by SELDI (see below). The use of mass spectrometry is especially attractive because it can distinguish and quantify modified forms of a protein based on mass and without the need for labeling.

Preferably, the biospecific capture reagent is bound to a solid phase, such as a bead, a plate, a membrane or a chip. Methods are well known for coupling biomolecules, such as by means of antibodies, to a solid phase. They can employ bifunctional linking agents, for example, or the solid phase can be derivatized with a reactive group, such as an epoxide or an imidizole, that will bind the molecule on contact. Biospecific capture reagents against different target proteins can be mixed in the same place, or they can be attached to solid phases in different physical or addressable locations.

For instance, one can load multiple columns with derivatized beads, each column able to capture a single protein cluster. Alternatively, one can pack a single column with different beads derivatized with capture reagents against a variety of protein clusters, thereby capturing all the analytes in a single place. Antibody-derivatized, bead-based technologies therefore can be used to detect the protein clusters. The biospecific capture reagents must be specifically directed toward the members of a cluster in order to differentiate them, however.

In yet another embodiment, the surface of a biochip (see below) can be derivatized with the capture reagents directed against protein clusters either in the same location or in physically different addressable locations on the biochip. One advantage of capturing different clusters in different addressable locations is that the analysis becomes simpler.

After identification of modified forms of a protein and correlation with the clinical parameter of interest, the modified form can be used as a biomarker in any of the methods of this invention. At this point, detection of the modified from can be accomplished by any specific detection methodology including affinity capture followed by mass spectrometry, or traditional immunoassay directed specifically to the modified form. Immunoassay requires biospecific capture reagents, such as antibodies, to capture the analytes. Furthermore, the assay must be designed to specifically distinguish a protein and modified forms of the protein. This can be done, for example, by employing a sandwich assay in which one antibody captures more than one form and second, distinctly labeled antibodies, specifically bind the various forms, thereby providing distinct detection of them. Antibodies can be produced by immunizing animals with the biomolecules. This invention contemplates traditional immunoassays including, for example, sandwich immunoassays including ELISA or fluorescence-based immunoassays, as well as other enzyme immunoassays.

Biomarkers and combinations of biomarkers identified in accordance with the present description may be used to qualify IHD risk in a patient. In particular, a biomarker or combination of biomarkers can be used to distinguish IHD patients from normal patients, and severe myocardial infarction from mild myocardial infarction, with a high degree of predictive success, i.e., greater than at least 85%, preferably greater than at least 90%, and more preferably greater than 95%.

The biomarkers can be resolved from other proteins in a sample by using a variety of fractionation techniques, e.g., chromatographic separation coupled with mass spectrometry, protein capture using immobilized antibodies or by traditional immunoassays. According to one aspect of the invention, the detection of biomarkers for diagnosis of ischaemic heart disease status entails contacting a sample from a subject with a substrate, e.g., beads or a SELDI probe, having an adsorbent thereon, under conditions that allow binding between the biomarker and the adsorbent, and then detecting the biomarker bound to the adsorbent by gas phase ion spectrometry, for example, mass spectrometry. Other detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

A “selective surface” is used to capture the biomarkers for analysis. The selective surface carriers an “adsorbent,” also called a “binding moiety” or “capture reagent.” An “adsorbent” or “capture reagent” or “binding moiety,” can be any material capable of binding an analyte. “Analyte” refers to any component of a sample that is desired to be detected. The term can refer to a single component or a plurality of components in the sample. The biomarkers of the invention are analytes. These may be attached directly to the substrate of the selective surface, or the substrate may be a “reactive surface” that carries a “reactive moiety” that is capable of binding the capture reagent, e.g., through a reaction forming a covalent or coordinate covalent bond. The phrase “reactive moiety” here denotes a chemical moiety that is capable of binding a capture reagent. Epoxide and carbodiimidizole are useful reactive moieties to covalently bind polypeptide capture reagents such as antibodies or cellular receptors. Nitriloacetic acid and iminodiacetic acid are useful reactive moieties that function as chelating agents to bind metal ions that interact non-covalently with histidine containing peptides. Some suitable adsorbents for use in SELDI, according to the invention, are described in U.S. Pat. No. 6,225,047.

“Chromatographic adsorbent” refers to a material typically used in chromatography. Chromatographic adsorbents include, for example, ion exchange materials, metal chelators (e.g., nitriloacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents). “Biospecific adsorbent” refers an adsorbent comprising a biomolecule, e.g., a nucleic. acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). In certain instances the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. Further examples of adsorbents for use in SELDI can be found in U.S. Pat. No. 6,225,047. “Adsorption” refers to detectable non-covalent binding of an analyte to an adsorbent or capture reagent.

A biospecific adsorbent is particularly useful in binding the galectin and fibrinogen biomarkers described herein. More particularly, both anti-galectin-3 antibodies and anti-troponin I antibodies retain significant amounts of biomarkers with very low non-specific capture of other proteins from sera using Ciphergen7 reactive adsorbent and Proteinchip7 arrays. Particular antibodies include anti-troponin I mouse monoclonal antibody clone 817 (Spectral Diagnosis7) and epitope 137aa to 148aa anti-galectin-3 mouse monoclonal antibody IgG1 (Affinity Bioreagents7).

In one aspect, the markers of this invention are detected by gas phase ion spectrometry, which involves the use of a gas phase ion spectrometer to detect gas phase ions. A “gas phase ion spectrometer” is an apparatus that detects gas phase ions. Gas phase ion spectrometers include an ion source that supplies gas phase ions. Gas phase ion spectrometers include, for example, mass spectrometers, ion mobility spectrometers, and total ion current measuring devices. “Gas phase ion spectrometry” refers to the use of a gas phase ion spectrometer to detect gas phase ions.

“Mass spectrometer” refers to a gas phase ion spectrometer that measures a parameter which can be translated into mass-to-charge ratios of gas phase ions. Mass spectrometers generally include an ion source and a mass analyzer. Examples of mass spectrometers are time-of-flight, magnetic sector, quadrupole filter, ion trap, ion cyclotron resonance, electrostatic sector analyzer and hybrids of these. “Mass spectrometry” refers to the use of a mass spectrometer to detect gas phase ions. “Laser desorption mass spectrometer” refers to a mass spectrometer which uses laser as a means to desorb, volatilize, and ionize an analyte. “Tandem mass spectrometer” refers to any mass spectrometer that is capable of performing two successive stages of m/z-based discrimination or measurement of ions, including ions in an ion mixture. The phrase includes mass spectrometers having two mass analyzers that are capable of performing two successive stages of m/z-based discrimination or measurement of ions tandem-in-space. The phrase further includes mass spectrometers having a single mass analyzer that is capable of performing two successive stages of m/z-based discrimination or measurement of ions tandem-in-time. The phrase thus explicitly includes Qq-TOF mass spectrometers, ion trap mass spectrometers, ion trap-TOF mass spectrometers, TOF-TOF mass spectrometers, Fourier transform ion cyclotron resonance mass spectrometers, electrostatic sector—magnetic sector mass spectrometers, and combinations thereof.

“Mass analyzer” refers to a sub-assembly of a mass spectrometer that comprises means for measuring a parameter which can be translated into mass-to-charge ratios of gas phase ions. In a time-of flight mass spectrometer the mass analyzer comprises an ion optic assembly, a flight tube and an ion detector.

“Ion source” refers to a sub-assembly of a gas phase ion spectrometer that provides gas phase ions. In one embodiment, the ion source provides ions through a desorption/ionization process. Such embodiments generally comprise a probe interface that positionally engages a probe in an interrogatable relationship to a source of ionizing energy (e.g., a laser desorption/ionization source) and in concurrent communication at atmospheric or subatmospheric pressure with a detector of a gas phase ion spectrometer.

Forms of ionizing energy for desorbing/lionizing an analyte from a solid phase include, for example: (1) laser energy; (2) fast atoms (used in fast atom bombardment); (3) high energy particles generated via beta decay of radionuclides (used in plasma desorption); and (4) primary ions generating secondary ions (used in secondary ion mass spectrometry). The preferred form of ionizing energy for solid phase analytes is a laser (used in laser desorption/ionization), in particular, nitrogen lasers, Nd-Yag lasers and other pulsed laser sources. “Fluence” refers to the laser energy delivered per unit area of interrogated image. Typically, a sample is placed on the surface of a probe, the probe is engaged with the probe interface and the probe surface is struck with the ionizing energy. The energy desorbs analyte molecules from the surface into the gas phase and ionizes them.

Other forms of ionizing energy for analytes include, for example: (1) electrons which ionize gas phase neutrals; (2) strong electric field to induce ionization from gas phase, solid phase, or liquid phase neutrals; and (3) a source that applies a combination of ionization particles or electric fields with neutral chemicals to induce chemical ionization of solid phase, gas phase, and liquid phase neutrals.

A preferred mass spectrometric technique for use in the invention is “Surface Enhanced Laser Desorption and Ionization” or “SELDI,” as described, for example, in U.S. Pat. Nos. 5,719,060 and U.S. Pat. No. 6,225,047, both to Hutchens and Yip. This refers to a method of desorption/ionization gas phase ion spectrometry (e.g., mass spectrometry) in which an analyte (here, one or more of the biomarkers) is captured on the surface of a SELDI probe that engages the probe interface of the gas phase ion spectrometer. In “SELDI MS,” the gas phase ion spectrometer is a mass spectrometer. One version of SELDI is called Surface-Enhanced Affinity Capture” or “SEAC.” This involves the use of probes comprised of an absorbent surface (a “SEAC probe”). In this context, “probe” refers to a device adapted to engage a probe interface and to present an analyte to ionizing energy for ionization and introduction into a gas phase ion spectrometer, such as a mass spectrometer. A probe typically includes a solid substrate, either flexible or rigid, that has a sample-presenting surface, on which an analyte is presented to the source of ionizing energy.

Another version of SELDI is Surface-Enhanced Neat Desorption (SEND), which involves the use of probes comprising energy absorbing molecules that are chemically bound to the probe surface (“SEND probe”). The phrase “Energy absorbing molecules” (EAM) denotes molecules that are capable of absorbing energy from a laser desorption/ionization source and, thereafter, contributing to desorption and ionization of analyte molecules in contact therewith. The EAM category includes molecules used in MALDI, frequently referred to as “matrix,” and is exemplified by cinnamic acid derivatives, sinapinic acid (SPA), cyano-hydroxy-cinnamic acid (CHCA) and dihydroxybenzoic acid, ferulic acid, and hydroxyaceto-phenone derivatives. In certain embodiments, the energy absorbing molecule is incorporated into a linear or cross-linked polymer, e.g., a polymethacrylate. For example, the composition can be a co-polymer of α-cyano-4-methacryloyloxycinnamic acid and acrylate. In another embodiment, the composition is a co-polymer of α-cyano-4-methacryloyloxycinnamic acid, acrylate and 3-(tri-methoxy)silyl propyl methacrylate. In another embodiment, the composition is a co-polymer of α-cyano-4-methacryloyloxycinnamic acid and octadecylmethacrylate (“C18 SEND”). SEND is further described in U.S. Pat. No. 5,719,060.

Another version of SELDI, called Surface-Enhanced Photolabile Attachment and Release (SEPAR), involves the use of probes having moieties attached to the surface that can covalently bind an analyte, and then release the analyte through breaking a photolabile bond in the moiety after exposure to light, e.g., to laser light. For instance, see U.S. Pat. No. 5,719,060. SEPAR and other forms of SELDI are readily adapted to detecting a biomarker or biomarker profile, pursuant to the present invention.

The detection of the biomarkers according to the invention can be enhanced by using certain selectivity conditions, e.g., adsorbents or washing solutions. The phrase “eluant” or “wash solution” refers to an agent, typically a solution, which is used to affect or modify adsorption of an analyte to an adsorbent surface and/or to remove unbound materials from the surface. The elution characteristics of a wash solution can depend, for example, on pH, ionic strength, hydrophobicity, degree of chaotropism, detergent strength, and temperature.

Pursuant to one aspect of the present invention, a sample is analyzed by means of a “biochip,” a term that denotes a solid substrate, having a generally planar surface, to which a capture reagent (adsorbent) is attached. Frequently, the surface of a biochip comprises a plurality of addressable locations, each of which has the capture reagent bound there. A biochip can be adapted to engage a probe interface and, hence, function as a probe in gas phase ion spectrometry preferably mass spectrometry. Alternatively, a biochip of the invention can be mounted onto another substrate to form a probe that can be inserted into the spectrometer.

“Protein biochip” refers to a biochip adapted for the capture of polypeptides. Many protein biochips are described in the art. These include, for example, protein biochips produced by Ciphergen Biosystems (Fremont, Calif.), Packard BioScience Company (Meriden Conn.), Zyomyx (Hayward, Calif.) and Phylos (Lexington, Mass.). Examples of such protein biochips are described in the following patents or patent applications: U.S. Pat. No. 6,225,047; PCT application WO 99/51773; U.S. Pat. No. 6,329,209, and PCT application WO 00/56934.

Protein biochips produced by Ciphergen Biosystems comprise surfaces having chromatographic or biospecific adsorbents attached thereto at addressable locations. Ciphergen ProteinChip® arrays include NP20, H4, H50, SAX-2, Q-10, WCX-2, CM-10, IMAC-3, IMAC-30, LSAX-30, LWCX-30, IMAC-40, PS-10, PS-20 and PG-20. These protein biochips comprise an aluminum substrate in the form of a strip. The surface of the strip is coated with silicon dioxide.

In the case of the NP-20 biochip, silicon oxide functions as a hydrophilic adsorbent to capture hydrophilic proteins. H4, H50, SAX-2, Q-10, WCX-2, CM-10, IMAC-3, IMAC-30, PS-10 and PS-20 biochips further comprise a functionalized, cross-linked polymer in the form of a hydrogel physically attached to the surface of the biochip or covalently attached through a silane to the surface of the biochip. The H4 biochip has isopropyl functionalities for hydrophobic binding. The H50 biochip has nonylphenoxy-poly(ethylene glycol)methacrylate for hydrophobic binding. The SAX-2 and Q-10 biochips have quaternary ammonium functionalities for anion exchange. The WCX-2 and CM-10 biochips have carboxylate functionalities for cation exchange. The IMAC-3 and IMAC-30 biochips have nitriloacetic acid functionalities that adsorb transition metal ions, such as Cu⁺⁺ and Ni⁺⁺, by chelation. These immobilized metal ions allow adsorption of peptide and proteins by coordinate bonding. The PS-10 biochip has carboimidizole functional groups that can react with groups on proteins for covalent binding. The PS-20 biochip has epoxide functional groups for covalent binding with proteins. The PS-series biochips are useful for binding biospecific adsorbents, such as antibodies, receptors, lectins, heparin, Protein A, biotin/streptavidin and the like, to chip surfaces where they function to specifically capture analytes from a sample. The PG-20 biochip is a PS-20 chip to which Protein G is attached. The LSAX-30 (anion exchange), LWCX-30 (cation exchange) and IMAC-40 (metal chelate) biochips have functionalized latex beads on their surfaces. Such biochips are further described, for example, in PCT publications WO 00/66265 and WO 00/67293, U.S. Pat. No. 6,555,813, and published U.S. patent application US 2003 0032043 A1.

Other Ciphergen ProteinChip® arrays comprise biospecific adsorbents or capture reagents, particularly antibodies. In one embodiment, antibodies are immobilized on the ProteinChip activated with 1,1′-carbonyldiimidazole (CDI) and stored in dry DMSO-acetone-acetic acid. The ProteinChip is extensively washed with deionized water right before antibody immobilization. The same technique can be used to activate the surface of passivated zirconia porous beads for subsequent antibody attachment. The antibodies, such as the anti-galectin-3 antibodies and anti-troponin I antibodies described above, then are immobilized on the CDI activated surface.

In keeping with the above-described principles, a substrate with an adsorbent is contacted with the sample, containing serum, for a period of time sufficient to allow biomarker that may be present to bind to the adsorbent. In one embodiment of the invention, more than one type of substrate with adsorbent thereon is contacted with the biological sample. For example, a sample may be applied to both a chip having an immobilized anti-galectin-3 antibody and a chip having an immobilized anti-troponin I antibody. This technique can allow for even more definitive assessment of patient status. After the incubation period, the substrate is washed to remove unbound material. Any suitable washing solutions can be used; preferably, aqueous solutions are employed.

An energy absorbing molecule then is applied to the substrate with the bound biomarkers. As noted, an energy absorbing molecule is a molecule that absorbs energy from an energy source such as a laser, thereby assisting in desorption of biomarkers from the substrate. Exemplary energy absorbing molecules include, as noted above, cinnamic acid derivatives, sinapinic acid and dihydroxybenzoic acid. Preferably sinapinic acid is used.

The biomarkers bound to the substrates are detected in a gas phase ion spectrometer such as a time-of-flight mass spectrometer. The biomarkers are ionized by an ionization source such as a laser, the generated ions are collected by an ion optic assembly, and then a mass analyzer disperses and analyzes the passing ions. The detector then translates information of the detected ions into mass-to-charge ratios. Detection of a biomarker typically will involve detection of signal intensity. Thus, both the quantity and mass of the biomarker can be determined.

Data generation in mass spectrometry begins with the detection of ions by the ion detector. A typical laser desorption mass spectrometer can employ a nitrogen laser at 337.1 nm. A useful pulse width is about 4 nanoseconds. Generally, power output of about 1-25 μJ is used. Ions that strike the detector generate an electric potential that is digitized by a high speed time-array recording device that digitally captures the analog signal. Ciphergen's ProteinChip® system employs an analog-to-digital converter (ADC) to accomplish this. The ADC integrates detector output at regularly spaced time intervals into time-dependent bins. The time intervals typically are one to four nanoseconds long. Furthermore, the time-of-flight spectrum ultimately analyzed typically does not represent the signal from a single pulse of ionizing energy against a sample, but rather the sum of signals from a number of pulses. This reduces noise and increases dynamic range. This time-of-flight data is then subject to data processing. In Ciphergen's ProteinChip® software, data processing typically includes TOF-to-M/Z transformation, baseline subtraction, high frequency noise filtering.

TOF-to-M/Z transformation involves the application of an algorithm that transforms times-of-flight into mass-to-charge ratio (M[Z). In this step, the signals are converted from the time domain to the mass domain. That is, each time-of-flight is converted into mass-to-charge ratio, or M/Z. Calibration can be done internally or externally. In internal calibration, the sample analyzed contains one or more analytes of known M/Z. Signal peaks at times-of-flight representing these massed analytes are assigned the known M/Z. Based on these assigned M/Z ratios, parameters are calculated for a mathematical function that converts times-of-flight to M/Z. In external calibration, a function that converts times-of-flight to M/Z, such as one created by prior internal calibration, is applied to a time-of-flight spectrum without the use of internal calibrants.

Baseline subtraction improves data quantification by eliminating artificial, reproducible instrument offsets that perturb the spectrum. It involves calculating a spectrum baseline using an algorithm that incorporates parameters such as peak width, and then subtracting the baseline from the mass spectrum.

High frequency noise signals are eliminated by the application of a smoothing function. A typical smoothing function applies a moving average function to each time-dependent bin. In an improved version, the moving average filter is a variable width digital filter in which the bandwidth of the filter varies as a function of, e.g., peak bandwidth, generally becoming broader with increased time-of-flight. See, e.g., WO 00/70648, Nov. 23, 2000 (Gavin et al., “Variable Width Digital Filter for Time-of-flight Mass Spectrometry”).

Data generated by desorption and detection of biomarkers can be analyzed with the use of a programmable digital computer. The computer program analyzes the data to indicate the number of markers detected, and optionally the strength of the signal and the determined molecular mass for each biomarker detected. Data analysis can include steps of determining signal strength of a biomarker and removing data deviating from a predetermined statistical distribution. For example, the observed peaks can be normalized, by calculating the height of each peak relative to some reference. The reference can be background noise generated by the instrument and chemicals such as the energy absorbing molecule which is set as zero in the scale.

The computer can transform the resulting data into various formats for display. The standard spectrum can be displayed, but in one useful format only the peak height and mass information are retained from the spectrum view, yielding a cleaner image and enabling biomarkers with nearly identical molecular weights to be more easily seen. In another useful format, two or more spectra are compared, conveniently highlighting unique biomarkers and biomarkers that are up- or down-regulated between samples. Using any of these formats, one can readily determine whether a particular biomarker is present in a sample.

In one format, referred to as “spectrum view or retentate map,” a standard spectral view can be displayed, wherein the view depicts the quantity of analyte reaching the detector at each particular molecular weight. In another format, referred to as “peak map,” only the peak height and mass information are retained from the spectrum view, yielding a cleaner image and enabling analytes with nearly identical molecular weights to be more easily seen. In yet another format, referred to as “gel view,” each mass from the peak view can be converted into a grayscale image based on the height of each peak, resulting in an appearance similar to bands on electrophoretic gels. In yet another format, referred to as “3-D overlays,” several spectra can be overlaid to study subtle changes in relative peak heights. In yet another format, referred to as “difference map view,” two or more spectra can be compared, conveniently highlighting unique analytes and analytes that are up- or down-regulated between samples.

Analysis generally involves the identification of peaks in the spectrum that represent signal from an analyte. Peak selection can, of course, be done by eye. However, software is available as part of Ciphergen's ProteinChip® software that can automate the detection of peaks. In general, this software functions by identifying signals having a signal-to-noise ratio above a selected threshold and labeling the mass of the peak at the centroid of the peak signal. In one useful application many spectra are compared to identify identical peaks present in some selected percentage of the mass spectra. One version of this software clusters all peaks appearing in the various spectra within a defined mass range, and assigns a mass (M/Z) to all the peaks that are near the mid-point of the mass (M/Z) cluster.

Software used to analyze the data can include code that applies an algorithm to the analysis of the signal to determine whether the signal represents a peak in a signal that corresponds to a biomarker according to the present invention. The software also can subject the data regarding observed biomarker peaks to classification tree or ANN analysis, to determine whether a biomarker peak or combination of biomarker peaks is present that indicates ischaemic heart disease status. Analysis of the data may be “keyed” to a variety of parameters that are obtained, either directly or indirectly, from the mass spectrometric analysis of the sample. These parameters include but are not limited to the presence or absence of one or more peaks, the shape of a peak or group of peaks, the height of one or more peaks, the log of the height of one or more peaks, and other arithmetic manipulations of peak height data.

In another aspect, the present invention provides kits for aiding in the diagnosis of ischaemic heart disease status, which kits are used to detect biomarkers according to the invention. The kits screen for the presence of biomarkers and combinations of biomarkers that are differentially present in samples from normal subjects and subjects with ischaemic heart disease. Measurement of one or more protein biomarkers using the kit, is by mass spectrometry or immunoassays such as an ELISA.

In one embodiment, the kit comprises a substrate having an adsorbent thereon, wherein the adsorbent is suitable for binding a biomarker according to the invention, and a washing solution or instructions for making a washing solution, in which the combination of the adsorbent and the washing solution allows detection of the biomarker using gas phase ion spectrometry, e.g., mass spectrometry. The kit may include more than type of adsorbent, each present on a different substrate. The detection of the biomarkers can be enhanced by using certain selectivity conditions, e.g., adsorbents or washing solutions.

In another embodiment, a kit of the invention may include a first substrate, comprising an adsorbent thereon, and a second substrate onto which the first substrate is positioned to form a probe, which can be inserted into a gas phase ion spectrometer, e.g., a mass spectrometer. In another embodiment, an inventive kit may comprise a single substrate that can be inserted into the spectrometer.

In a further embodiment, such a kit can comprise instructions for suitable operational parameters in the form of a label or separate insert. For example, the instructions may inform a consumer how to collect the sample or how to wash the probe. In yet another embodiment the kit can comprise one or more containers with biomarker samples, to be used as standard(s) for calibration.

In a preferred embodiment, the detection of biomarkers for diagnosis of ischaemic heart disease in a subject entails contacting a sample from a subject or patient, preferably a serum sample, with a substrate having an adsorbent thereon under conditions that allow binding between the biomarker and the adsorbent, and then detecting the biomarker bound to the adsorbent by gas phase ion spectrometry, preferably by Surface Enhanced Laser Desorption/Ionization (SELDI) mass spectrometry. The biomarkers are ionized by an ionization source such as a laser. The generated ions are collected by an ion optic assembly and accelerated toward an ion detector. Ions that strike the detector generate an electric potential that is digitized by a high speed time-array recording device that digitally captures the analog signal. Ciphergen's ProteinChip® system employs an analog-to-digital converter (ADC) to accomplish this. The ADC integrates detector output at regularly spaced time intervals into time-dependent bins. The time intervals typically are one to four nanoseconds long. Furthermore, the time-of-flight spectrum ultimately analyzed typically does not represent the signal from a single pulse of ionizing energy against a sample, but rather the sum of signals from a number of pulses. This reduces noise and increases dynamic range. This time-of-flight data is then subject to data processing. In Ciphergen's ProteinChip® software, data processing typically includes TOF-to-M/Z transformation, baseline subtraction, high frequency noise filtering. Thus, both the quantity and mass of the biomarker can be determined.

In some embodiments, data derived from the spectra (e.g., mass spectra or time-of-flight spectra) that are generated using samples such as “known samples” can then be used to “train” a classification model. A “known sample” is a sample that is pre-classified. The data that are derived from the spectra and are used to form the classification model can be referred to as a “training data set.” Once trained, the classification model can recognize patterns in data derived from spectra generated using unknown samples. The classification model can then be used to classify the unknown samples into classes. This can be useful, for example, in predicting whether or not a particular biological sample is associated with a certain biological condition (e.g., diseased versus non diseased).

The training data set that is used to form the classification model may comprise raw data or pre-processed data. In some embodiments, raw data can be obtained directly from time-of-flight spectra or mass spectra, and then may be optionally “pre-processed” as described above.

Classification models can be formed using any suitable statistical classification (or “learning”) method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Patern Analysis and Machine Intelligence, Vol. 22, No. 1, January 2000.

In supervised classification, training data containing examples of known categories are presented to a learning mechanism, which learns one more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART—classification and regression trees), artificial neural networks such as back propagation networks, discriminant analyses (e.g., Bayesian classifier or Fischer analysis), logistic classifiers, and support vector classifiers (support vector machines).

A preferred supervised classification method is a recursive partitioning process. Recursive partitioning processes use recursive partitioning trees to classify spectra derived from unknown samples. Further details about recursive partitioning processes are provided in U.S. 2002 0138208 A1 (Paulse et al., “Method for analyzing mass spectra,” Sep. 26, 2002.

In other embodiments, the classification models that are created can be formed using unsupervised learning methods. Unsupervised classification attempts to learn classifications based on similarities in the training data set, without pre classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described in, for example, WO 01/31580 (Barnhill et al., “Methods and devices for identifying patterns in biological systems and methods of use thereof,” May 3, 2001); U.S. 2002 0193950 A1 (Gavin et al., “Method or analyzing mass spectra,” Dec. 19, 2002); U.S. 2003 0004402 A1 (Hitt et al., “Process for discriminating between biological states based on hidden patterns from biological data,” Jan. 2, 2003); and U.S. 2003 0055615 A1 (Zhang and Zhang, “Systems and methods for processing biological expression data” Mar. 20, 2003).

The classification models can be formed on and used on any suitable digital computer. Suitable digital computers include micro, mini, or large computers using any standard or specialized operating system such as a Unix, Windows™ or Linux™ based operating system. The digital computer that is used may be physically separate from the mass spectrometer that is used to create the spectra of interest, or it may be coupled to the mass spectrometer.

The training data set and the classification models according to embodiments of the invention can be embodied by computer code that is executed or used by a digital computer. The computer code can be stored on any suitable computer readable media including optical or magnetic disks, sticks, tapes, etc., and can be written in any suitable computer programming language including C, C++, visual basic, etc.

Armed with the information regarding the biomarkers identified herein, one can use various methods to recognize biomarkers according to the invention. These methods take raw data, regarding which peaks are present and their intensity, and provide a differential diagnosis of ischaemic heart disease versus normal, and of severe versus mild myocardial infarction, for a sample.

Determination of Subject IHD Status

Any biomarker, individually, is useful in aiding in the determination of IHD status. First, the selected biomarker is measured in a subject sample using the methods described herein, e.g., capture on a SELDI biochip followed by detection by mass spectrometry. Then, the measurement is compared with a diagnostic amount or control that distinguishes an IHD status from a non-IHD status. The diagnostic amount will reflect the information herein that a particular biomarker is up-regulated or down-regulated in an IHD status compared with a non-IHD status. As is well understood in the art, the particular diagnostic amount used can be adjusted to increase sensitivity or specificity of the diagnostic assay depending on the preference of the diagnostician. The test amount as compared with the diagnostic amount thus indicates IHD status.

While individual biomarkers are useful diagnostic markers, it has been found that a combination of biomarkers provides greater predictive value than single markers alone. Specifically, the detection of a plurality of markers in a sample increases the percentage of true positive and true negative diagnoses and decreases the percentage of false positive or false negative diagnoses. Thus, preferred methods of the present invention comprise the measurement of more than one biomarker. In particular, a combination of galectin-3 and fibrinogen biomarkers is able to distinguish severe myocardial infarction from mild myocardial infarction. More particularly, the combination of a 7.8 kDa peak associated with a galectin-3 biomarker and one or both of 6.7 kDa and 8.9 kDa peaks associated with fibrinogen biomarkers, are useful combinations. Further useful combinations of peaks are those produced by fragments of the biomarkers that correspond to the 7.8, 6.7 and 8.9 kDa peaks, respectively.

In some embodiments, the mere presence or absence of a marker, without quantifying the amount of marker, is useful and can be correlated with a probable diagnosis of IHD. For example, galectin-3 and fibrinogen biomarkers can be more frequently detected in human IHD patients than in normal subjects. Thus, a detected presence or absence, respectively, of these markers in a subject being tested indicates that the subject has a higher probability of having IHD.

In other embodiments, the measurement of markers can involve quantifying the markers to correlate the detection of markers with a probable diagnosis of IHD. Thus, if the amount of the markers detected in a subject being tested is different compared to a control amount (i.e., higher or lower than the control, depending on the marker), then the subject being tested has a higher probability of having IHD.

The correlation may take into account the amount of the marker or markers in the sample compared to a control amount of the marker or markers (up or down regulation of the marker or markers) (e.g., in normal subjects in whom heart disease is undetectable). A control can be, e.g., the average or median amount of marker present in comparable samples of normal subjects in whom heart disease is undetectable. One or more controls also can be packaged with a kit used to determine IHD status, to be run in parallel with the sample taken from the patient.

In certain embodiments of the methods of qualifying IHD status, the methods further comprise managing subject treatment based on the status. As aforesaid, such management describes the actions of the physician or clinician subsequent to determining IHD status. For example, if the result of the methods of the present invention is inconclusive or there is reason that confirmation of status is necessary, the physician may order more tests. Alternatively, if the status indicates that surgery is appropriate, the physician may schedule the patient for surgery. In other instances, the patient may receive treatment with diet or with various heart medications that are well known in the art, such as TPA, ACE inhibitors, beta blockers, nitroglycerin, cholesterol-lowering drugs, blood thinners, aspirin, and diuretics, either in lieu of, or in addition to, surgery. Furthermore, if the results show that treatment has been successful, no further management may be necessary.

The methods of the present invention have other applications as well. For example, the markers can be used to screen for compounds that modulate the expression of the markers in vitro or in vivo, which compounds in turn may be useful in treating or preventing IHD in patients. In another example, the markers can be used to monitor the response to treatments for IHD. In yet another example, the markers can be used in heredity studies to determine if the subject is at risk for developing IHD.

The contents of each document mentioned in the application are incorporated herein in their entirety by reference.

The following examples are offered by way of illustration, and are not limiting.

### EXAMPLE 1.

### Analysis of Serum Samples to Reveal Potential Protein Biomarkers

Passivated zirconia porous beads activated via 1,1′ carbonyl diimidazole (CDI) and stored in dry DMSO-acetone-acetic acid were used to immobilize various antibodies. Right before antibody immobilization, the beads were washed extensively in de-ionized water.

Three antibodies were used, anti troponin I mouse monoclonal antibody clone 817 (Spectral Diagnosis7), epitope 137aa to 148aa, anti Galectin-3 mouse monoclonal antibody IgG1 (Affinity Bioreagents7), and mouse IgG (mlgG) as a control antibody (Sigma7). They were brought into contact with CDI reactive beads at 0.5 mg/mL concentration, and at two times beads volume. Antibody was coupled at 4° C. for 16 hours.

Human serum samples derived from either normal patients, or patients suffering from both mild and severe ischaemia, were then incubated with beads for 1 hour. After washing, the captured material was eluted using 1% trifluoroacetic acid (TFA), and analyzed directly by surface enhanced laser desorption ionization mass spectrometry (SELDI-MS) using a Ciphergen7 PBS IIc, a laser desorption/ionization, time-lag focusing, time-of-flight mass spectrometer (TOFMS).

Serum samples from different patients were analyzed, peak patterns were generated through parallel comparison among the same class of patient samples. FIGS. 1A and 1B show biomarker patterns for samples from subjects with severe and mild ischaemic infarction, respectively. Samples from patients with severe myocardial infarction had and intense protein peak at 7.8 kDa, while samples from patients with mild myocardial infarction had intense protein peaks at 6.7 and 8.9 kDa, respectively.

The patterns shown in FIGS. 1A and 1B were initially generated via anti-troponin 817 antibody reaction with patient sera. After the 7.8 kDa peak was identified as galectin-3, an anti-galectin-3 mouse monoclonal antibody was used, and generated an identical capture pattern.

### EXAMPLE 2

### Analysis and Identification of Potential Protein Biomarkers

Proteins corresponding to the three peaks identified in Example 1 were subjected to various purification schemes for enrichment, enzymatic digestion, and quadruple mass sequencing using an Applied Biosystems/Sciex QStar® equipped with a Ciphergen ProteinChip® Interface.

Mass sequencing of the enzymatic digest of the 7.8 kDa peak produced the peptide fingerprint shown in FIG. 2. Fragmented ions from the 1985 peptide of FIG. 2 produced an even distribution of ions ranging in size from 175 to 1300 Da, as shown in FIG. 3. This even distribution assured a good score in protein matching during a Mascot database search. A score of 47 was obtained for the Mascot search. Since all scores greater than 35 were highly significant, this result, coupled with good separation from other matches, confirmed that the 1985 peptide is galectin-3. Searches in other databases also identified the 7.8 kDa peak and the 1985 peptide fingerprint as being galectin-3 .

Mass sequencing of enzymatic digests of the 6.7 and 8.9 kDa peaks produced the peptide fingerprint shown in FIG. 4. Ionized fragments from each of the peptides indicated with an asterisk (*) in FIG. 4 produced an even distribution of ions, ranging in size from 900 to 2300 Da, as shown in FIG. 5. The peptides in the range of 2100 Da to 2300 Da are largely from trypsin and were excluded from mass sequencing. Major peptide peaks are indicated with an asterisk.

Fragmented ions from the 1887 peptide are shown in FIG. 5, and were subjected to a Mascot database search. A score of 26 was obtained for the Mascot search. Since all scores greater than 16 were highly significant, this result, coupled with good separation from other matches, confirmed that the 1887 peptide is fibrinogen. Searches in other databases also identified the 1887 peptide as fibrinogen. Besides the 1887 peptide, all other highlighted peptides, including 1032, 1552, 1637 and 1846, were mass sequenced and shown to be homologous to fibrinogen.

What is claimed is:

1. A method of qualifying ischaemic heart disease status in a subject comprising:
(a) measuring at least one biomarker in a sample from the subject, wherein the biomarker is selected from the group consisting of galectin-3 and galectin-3 fragments, and
(b) correlating the measurement with ischaemic heart disease status.

2. The method of claim 1, further comprising:
(c) managing subject treatment based on the status.

3. The method of claim 2, wherein managing subject treatment is selected from ordering more tests, performing surgery, prescribing medication, and taking no further action.

4. The method of claim 2, further comprising:
(d) measuring at least one biomarker after subject management.

5. The method of claim 1, wherein the ischaemic heart disease status is selected from the group consisting of the presence or absence of disease, the degree of disease and the effectiveness of treatment of disease.

6. The method of claim 5, further comprising measuring at least one additional biomarker selected from the group consisting of fibrinogen and fibrinogen fragments and correlating measurement of the biomarkers measured with ischaemic heart disease status.

7. The method of claim 1, wherein measuring comprises:
(a) providing a subject sample of blood or a blood derivative;
(b) fractionating proteins in the sample and collecting fractions that contain galectin-3 or a galectin-3 fragment biomarker; and
(c) capturing the biomarkers from the fractions on a surface of a substrate comprising capture reagents that bind the biomarkers.

8. The method of claim 7, wherein the substrate is a SELDI probe comprising an adsorbent that captures the biomarkers and wherein the biomarkers are detected by SELDI.

9. The method of claim 8, wherein the SELDI probe comprises a biospecific affinity reagent that binds the biomarkers.

10. The method of claim 7, wherein the substrate is a microtiter plate comprising biospecific affinity reagents that bind the biomarkers and the biomarkers are detected by immunoassay.

11. The method of claim 1, wherein measuring is selected from detecting the presence or absence of the biomarkers(s), quantifying the amount of marker(s), and qualifying the type of biomarker.

12. The method of claim 1, wherein at least one biomarker is measured using a biochip array.

13. The method of claim 12, wherein the biochip array is a protein chip array.

14. The method of claim 12, wherein at least one biomarker is immobilized on the biochip array.

15. The method of claim 1, wherein the biomarkers are measured by SELDI.

16. The method of claim 1, wherein the biomarkers are measured by immunoassay.

17. The method of claim 1, wherein the correlating is performed by a software classification algorithm.

18. The method of claim 1, wherein the sample is selected from blood, serum and plasma.

19. A method comprising measuring a plurality of biomarkers in a sample from the subject, wherein the biomarkers are selected from the group consisting of galectin-3 and galectin-3 fragments.

20. The method of claim 19, further comprising measuring an additional biomarker selected from the group consisting of fibrinogen and fibrinogen fragments.

21. The method of claim 20, wherein a plurality of the additional biomarkers are measured.

22. The method of claim 19, wherein the biomarkers are detected by SELDI or immunoassay.

23. The method of claim 19, wherein the sample is selected from blood, serum and plasma.

24. A method comprising measuring at least one biomarker in a sample from a subject, wherein the biomarker is selected from the group consisting of galectin-3 and galectin-3 fragments.

25. The method of claim 24, further comprising measuring an additional biomarker selected from the group consisting of fibrinogen and fibrinogen fragments.

26. The method of claim 25, wherein a plurality of the additional biomarkers are measured.

27. The method of claim 24, wherein the biomarkers are detected by SELDI or immunoassay.

28. The method of claim 24, wherein the sample is selected from blood, serum and plasma.

29. A kit comprising:
(a) a capture reagent that binds a biomarker selected from galectin-3 and galectin-3 fragments; and
(b) instructions for using the capture reagent to detect the biomarker.

30. The kit of claim 29, additionally comprising:
(c) a container comprising at least one of the biomarkers.

31. The kit of claim 29, wherein the capture reagent binds a plurality of the biomarkers.

32. The kit of claim 29, wherein the capture reagent is a SELDI probe.

33. The kit of claim 29, further comprising an additional capture reagent that binds an additional biomarker selected from the group consisting of fibrinogen and fibrinogen fragments.

34. The kit of claim 33, wherein the additional capture reagent binds a plurality of the additional biomarkers.

35. The kit of claim 33, wherein the additional capture reagent is a SELDI probe.

36. The kit of claim 33, wherein the capture reagent is an antibody.

37. The kit of claim 36, wherein the capture reagent is an anti-galectin-3 antibody.

38. The kit of claim 36, wherein the capture reagent is an anti-troponin I antibody.

39. The kit of claim 33, wherein the additional capture reagent is an antibody.

40. The kit of claim 33, wherein the additional capture reagent is an anti-fibrinogen antibody.

41. The kit of claim 33, wherein the additional capture reagent is an anti-troponin I antibody.

42. The kit of claim 33, wherein the additional capture reagent is an anti-galectin-3 antibody.

43. The kit of claim 29, comprising a SELDI probe to which the capture reagent is attached or is attachable.

44. The kit of claim 33, comprising a SELDI probe to which the additional capture reagent is attached or is attachable.

45. The kit of claim 29, further comprising a wash solution that selectively allows retention of bound biomarker to the capture reagent as compared with other proteins after washing.

46. A kit comprising:
(a) a single capture reagent that binds both a biomarker selected from the group consisting of galectin-3 and galectin-3 fragments and an additional biomarker selected from the group consisting of fibrinogen and fibrinogen fragments; and
(b) instructions for using the capture reagent to detect both of the biomarkers.

47. The kit of claim 46, additionally comprising:
(c) a container comprising at least one of the biomarkers.

48. The kit of claim 46, additionally comprising:
(c) at least one container comprising at least on of the biomarkers; and
(d) at least one container comprising at least one of the additional biomarkers.

49. The kit of claim 46, wherein the capture reagent is an antibody.

50. The kit of claim 49, wherein the capture reagent is an anti-troponin antibody.

51. The kit of claim 49, wherein the capture reagent is an anti-galectin-3 antibody.

52. The kit of claim 46, comprising a SELDI probe to which the capture reagent is attached or is attachable.

53. The kit of claim 46, further comprising a wash solution that selectively allows retention of bound biomarkers to the capture reagent as compared with other proteins after washing.

54. The kit of claim 46, further comprising written instructions for use of the kit in the diagnosis of ischaemic heart disease.

55. The kit of claim 47, wherein the instructions provide for contacting the biomarker(s) in container (c) with the capture reagent and describe the result of the contacting.

56. The kit of claim 48, wherein the instructions provide for contacting the biomarkers in containers (c) and (d) with the capture reagent and describe the result of the contacting.
